(19) United States
(12) Patent Application Publication
(10) Pub. No.: US 2026/0165680 A1
(43) Pub. Date: Jun. 18, 2026

(54) ULTRASOUND IMAGING APPARATUS AND METHOD FOR DISPLAYING THREE-DIMENSIONAL ULTRASOUND IMAGE

(71) Applicant: Samsung Medison Co., Ltd., Hongcheon-gun (KR)

(72) Inventors: Sungwook Park, Seoul (KR); Jinyong Lee, Seoul (KR)

(73) Assignee: SAMSUNG MEDISON CO., LTD., Hongcheon-gun (KR)

(21) Appl. No.: 19/178,713

(22) Filed: Apr. 14, 2025

(30) Foreign Application Priority Data
Dec. 18, 2024 (KR) 10-2024-0190458

(51) Int. Cl. A61B 8/00; A61B 8/08; G06T 15/08

(52) U.S. Cl. CPC A61B 8/466; A61B 8/0883; A61B 8/483; A61B 8/54; G06T 15/08; G06T 2210/36; G06T 2210/41

(57) ABSTRACT

Provided are an ultrasound imaging apparatus and method for displaying a three-dimensional (3D) ultrasound image. In particular, the ultrasound imaging apparatus includes an ultrasound transmitter/receiver module configured to obtain 3D volume data for displaying the 3D ultrasound image, a display displaying the 3D ultrasound image, a memory storing at least one instruction, and at least one processor electrically connected to the ultrasound transmitter/receiver module, the display, and the memory, wherein the at least one processor is configured to execute the at least one instruction to cause the ultrasound imaging apparatus to obtain the 3D volume data, set at least two regions of interest (ROIs) in the 3D volume data, perform high-quality rendering on a first ROI among the at least two ROIs, and perform high-speed rendering on a second ROI other than the first ROI.

### CROSS-REFERENCE TO RELATED APPLICATION

This application is based on and claims priority under 35 U.S.C. § 119 to Korean Patent Application No. 10-2024-0190458, filed on Dec. 18, 2024, in the Korean Intellectual Property Office, the disclosure of which is incorporated by reference herein in its entirety.

### BACKGROUND

### 1. Field

The disclosure relates to an ultrasound imaging apparatus and method for displaying a three-dimensional (3D) ultrasound image. More particularly, the disclosure relates to a technique for setting properties for rendering 3D volume data and performing rendering according to the set properties in order to improve the display performance of a 3D ultrasound image.

### 2. Description of the Related Art

Recently, in the medical field, various types of medical imaging apparatuses have been widely used to visualize and obtain information about living tissue of a human body for early diagnosis or surgery with regard to various diseases. Representative examples of these medical imaging apparatuses may include an ultrasound imaging apparatus, a computed tomography (CT) apparatus, and a magnetic resonance imaging (MRI) apparatus.

Ultrasound imaging apparatuses transmit ultrasound signals generated by transducer elements of a probe to an object and receive information of signals reflected from the object, thereby non-invasively obtaining at least one image of an internal part (e.g., soft tissue or blood flow) of the object. Ultrasound imaging apparatuses are used for medical purposes including observing an internal area of an object, detecting foreign substances, assessing injuries, etc. Such ultrasound imaging apparatuses have the advantages of being highly stable, capable of displaying images in real time, and safe due to there being no radiation exposure, as compared to X-ray apparatuses, and therefore, have been widely used together with other types of imaging diagnostic apparatuses.

An ultrasound imaging apparatus is capable of displaying a three-dimensional (3D) ultrasound image. The ultrasound imaging apparatus may render 3D volume data to display the 3D ultrasound image. When rendering the 3D volume data, the ultrasound imaging apparatus may set properties for rendering the 3D volume data. For example, when rendering the 3D volume data, the ultrasound imaging apparatus may set transparency, a color map, a gamma curve, a post-gain, an image filter, etc. for rendering the 3D volume data.

When rendering 3D volume data to display a 3D ultrasound image by using a conventional ultrasound imaging apparatus, it is not easy to render portions corresponding to different regions in the 3D ultrasound image according to different properties. For example, when rendering 3D volume data by using a conventional ultrasound imaging apparatus, it is not easy to render a specific region (e.g., a region of interest (ROI)) at a high quality while rendering the remaining regions thereof at a high speed.

As a result, a 3D ultrasound image may be generated via rendering with one property even in a case where a specific region needs to be displayed in more detail than the remaining regions and the remaining regions may be displayed relatively briefly. In this case, the quality at which the specific region is displayed may be lower than the desired quality, or the remaining regions may be displayed in unnecessary detail, thus decreasing a display speed of the 3D ultrasound image.

### SUMMARY

Additional aspects will be set forth in part in the description which follows and, in part, will be apparent from the description, or may be learned by practice of the presented embodiments of the disclosure.

According to an embodiment, an ultrasound imaging apparatus for displaying a three-dimensional (3D) ultrasound image may include an ultrasound transmitter/receiver module configured to obtain 3D volume data for displaying the 3D ultrasound image, a display displaying the 3D ultrasound image, a memory storing at least one instruction, and at least one processor electrically connected to the ultrasound transmitter/receiver module, the display, and the memory, wherein the at least one processor is configured to execute the at least one instruction to cause the ultrasound imaging apparatus to obtain the 3D volume data, set at least two regions of interest (ROIs) in the 3D volume data, perform high-quality rendering on a first ROI among the at least two ROIs, and perform high-speed rendering on a second ROI other than the first ROI.

According to an embodiment, a method, performed by an ultrasound imaging apparatus, of displaying a 3D ultrasound image may include obtaining 3D volume data, setting at least two ROIs in the 3D volume data, performing high-quality rendering on a first ROI among the at least two ROIs, and performing high-speed rendering on a second ROI other than the first ROI.

According to an embodiment, a method, performed by an ultrasound imaging apparatus, of displaying a 3D ultrasound image may include obtaining 3D volume data, identifying at least one anatomical structure in the 3D volume data, setting the identified at least one anatomical structure as at least one ROI, and performing rendering on the at least one ROI based on a preset value.

### BRIEF DESCRIPTION OF THE DRAWINGS

The above and other aspects, features, and advantages of certain embodiments of the

disclosure will be more apparent from the following description taken in conjunction with the accompanying drawings, wherein reference numerals denote structural elements, and in which:

FIGS. 1A and 1B are block diagrams of configurations of an ultrasound imaging system according to an embodiment;

FIGS. 2A, 2B, 2C, and 2D are diagrams illustrating examples of an ultrasound imaging system according to an embodiment;

FIG. 3 illustrates a three-dimensional (3D) ultrasound image displayed by an ultrasound imaging apparatus, according to an embodiment;

FIG. 4 is a flowchart of a method, performed by an ultrasound imaging apparatus, of displaying a 3D ultrasound image, according to an embodiment;

FIG. 5 is a diagram illustrating an ultrasound imaging apparatus obtaining 3D volume data, according to an embodiment;

FIG. 6 is a diagram illustrating an ultrasound imaging apparatus setting a region of interest (ROI), according to an embodiment;

FIG. 7 is a diagram illustrating an ultrasound imaging apparatus controlling a speed at which an ROI is rendered, according to an embodiment;

FIG. 8 is a diagram illustrating an ultrasound imaging apparatus controlling a quality at which an ROI is rendered, according to an embodiment;

FIG. 9 is a diagram illustrating an ultrasound imaging apparatus individually controlling a speed and a quality at which an ROI is rendered;

FIG. 10 is a flowchart of a method, performed by an ultrasound imaging apparatus, of displaying a 3D ultrasound image, according to an embodiment;

FIG. 11 is a diagram illustrating an ultrasound imaging apparatus setting an anatomical structure as an ROI, according to an embodiment;

FIG. 12 is a flowchart of a method, performed by an ultrasound imaging apparatus, of displaying a 3D ultrasound image, according to an embodiment; and

FIG. 13 is a diagram illustrating an ultrasound imaging apparatus setting an abnormal structure as an ROI, according to an embodiment.

### DETAILED DESCRIPTION

The disclosure describes principles of embodiments of the disclosure and sets forth embodiments thereof to clarify the scope of the claims of the disclosure and to allow one of ordinary skill in the art to implement the embodiments. The embodiments may be implemented in various forms.

Like reference numerals refer to like elements throughout the specification. This specification does not describe all elements of the embodiments, and general knowledge in the art to which the disclosure belongs or descriptions overlapping between the embodiments will be omitted. As used in herein, the term “module” or “unit” may be implemented in one or a combination of two or more of software, hardware, or firmware, and in some embodiments, a plurality of “modules” or “units” may be implemented as a single element, or a single “module” or “unit” may include a plurality of elements.

A singular form of a noun corresponding to an item may include one or a plurality of the items unless the context clearly indicates otherwise.

As used herein, each of the phrases such as “A or B,” “at least one of A and B, “at least one of A or B,” “A, B, or C,” “at least one of A, B, and C,” and “at least one of A, B, or C” may include any one of the items listed together in a corresponding one of the phrases, or all possible combinations thereof.

The term “and/or” includes any combination of a plurality of associated elements listed, or any one of the plurality of associated listed elements.

Terms such as “first,” “second,” etc. may be used simply to distinguish an element from other elements and do not limit the elements in any other respect (e.g., importance or order).

Furthermore, as used in the disclosure, the terms “front,” “rear,” “top,” “bottom,” “side,” “left,” “right,” “upper,” “lower,” etc. are defined based on the drawings, and the shape and position of each component are not limited by these terms.

The terms such as “comprise,” “include,” or “have” are intended to specify the presence of stated features, numbers, steps, operations, elements, components, or combinations thereof, but do not preclude the presence or addition of one or more other features, numbers, steps, operations, elements, components, or combinations thereof.

It will also be understood that when an element is referred to as “connected,” “coupled,” “supported,” or “in contact” with another element, this includes not only when the elements are directly connected, coupled, supported, or in contact, but also when they are indirectly connected, coupled, supported, or in contact via a third element.

It will also be understood that when an element is referred to as being “on” another element, the element may be directly on the other element, or intervening elements may also be present therebetween.

Hereinafter, ultrasound apparatuses according to various embodiments will be described in detail with reference to the accompanying drawings. In the following description with reference to the accompanying drawings, identical or corresponding components are assigned like reference numbers, and redundant descriptions thereof may be omitted.

In the disclosure, an image may include a medical image obtained by a medical imaging apparatus such as a magnetic resonance imaging (MRI) apparatus, a computed tomography (CT) apparatus, an ultrasound imaging apparatus, or an X-ray apparatus.

As used herein, an ‘object’ is a target to be imaged, and may include a human, an animal, or a part thereof. For example, the object may include a part of a body (organ, tissue, or the like), or a phantom.

As used herein, an ‘ultrasound image’ refers to an image of an object generated or processed based on ultrasound signals transmitted to the object and reflected therefrom.

Hereinafter, embodiments of the disclosure are described in detail with reference to the drawings.

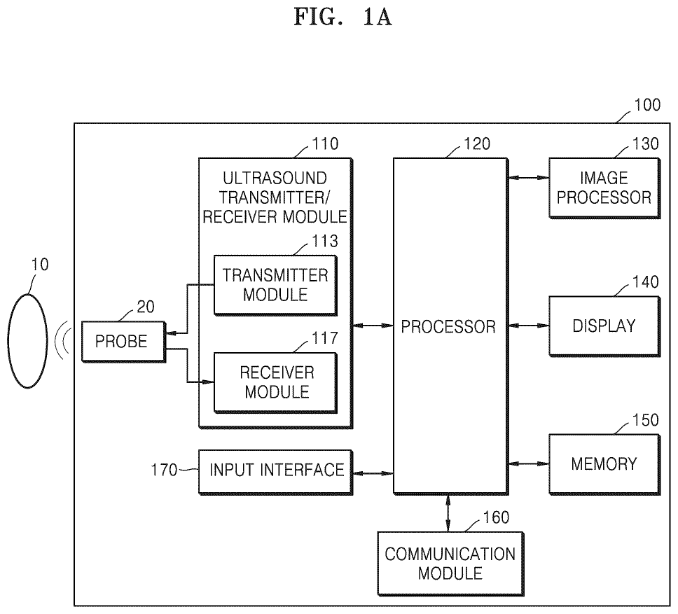

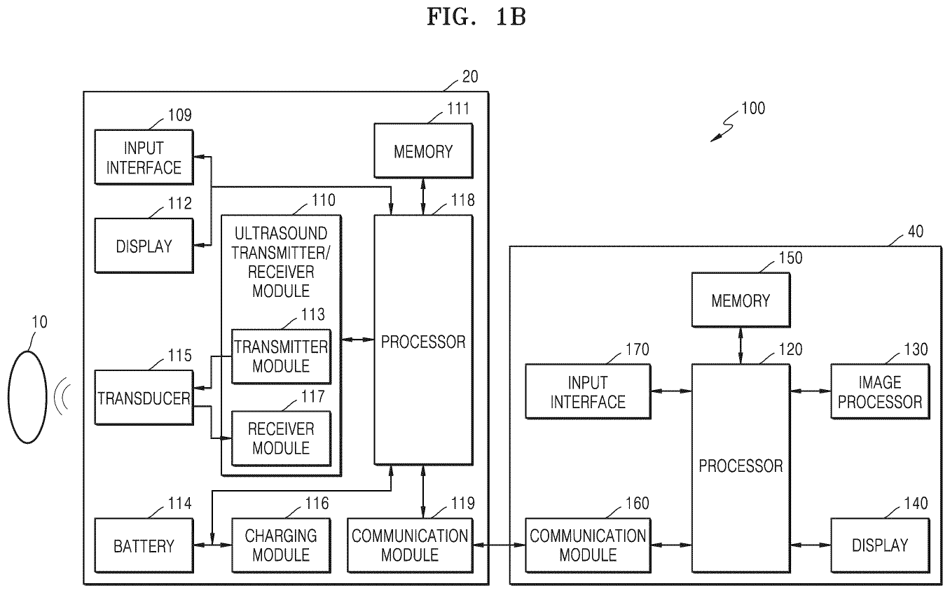

FIGS. 1A and 1B are block diagrams of configurations of an ultrasound imaging system according to an embodiment.

Referring to FIGS. 1A and 1B, an ultrasound imaging system 100 may include a probe 20 and an ultrasound imaging apparatus 40.

The ultrasound imaging apparatus 40 may be implemented not only as a cart-type ultrasound imaging apparatus but also as a portable ultrasound imaging apparatus. Examples of the portable ultrasound imaging apparatus may include, but are not limited to, a smartphone, a laptop computer, a personal digital assistant (PDA), a tablet personal computer (PC), etc., each of which includes a probe and an application. The ultrasound imaging apparatus 40 may be formed integrally with the probe 20.

The probe 20 may include a wired probe that is connected to the ultrasound imaging apparatus 40 by wire to communicate with the ultrasound imaging apparatus 40 by wire, a wireless probe that is wirelessly connected to the ultrasound imaging apparatus 40 to communicate wirelessly with the ultrasound imaging apparatus 40, and/or a hybrid probe that is connected to the ultrasound imaging apparatus 40 by wire or wirelessly to communicate with the ultrasound imaging apparatus 40 by wire or wirelessly.

According to various embodiments, the ultrasound imaging apparatus 40 may include an ultrasound transmitter/receiver module 110 as shown in FIG. 1A, or the probe 20 may include the ultrasound transmitter/receiver module 110 as shown in FIG. 1B. According to various embodiments, the ultrasound imaging apparatus 40 and the probe 20 may both include the ultrasound transmitter/receiver module 110.

According to various embodiments, the probe 20 may further include at least one of an image processor 130, a display 140, or an input interface 170, or a combination thereof. In the disclosure, descriptions of the ultrasound transmitter/receiver module 110, the image processor 130, the display 140, or the input interface 170 included in the ultrasound imaging apparatus 40 may also apply to the ultrasound transmitter/receiver module 110, the image processor 130, the display 140, or the input interface 170 included in the probe 20.

FIG. 1A is a block diagram of a configuration of the ultrasound imaging system 100 when the probe 20 is a wired probe or a hybrid probe.

The probe 20 may include a plurality of transducer elements. The plurality of transducer elements are arranged in a predetermined array, forming a transducer array. The transducer array may correspond to a one-dimensional (1D) array or a two-dimensional (2D) array. The plurality of transducer elements may transmit ultrasound signals to an object 10 in response to transmission signals applied from the transmitter module 113. The plurality of transducer elements may receive ultrasound (echo) signals reflected from the object 10 to form reception signals. Furthermore, the probe 20 may be formed integrally with the ultrasound imaging apparatus 40, or may be implemented as a separate part connected to the ultrasound imaging apparatus 40 in a wired manner. In addition, the ultrasound imaging apparatus 40 may be connected to one or a plurality of probes 20 according to its implemented configuration.

When the probe 20 is a wired probe or hybrid probe, the probe 20 may include a cable and a connector that are connectable to a connector of the ultrasound imaging apparatus 40.

According to an embodiment, the probe 20 may be implemented as a 2D probe. When the probe 20 is implemented as a 2D probe, the plurality of transducer elements included in the probe 20 may be arranged in two dimensions to form a 2D transducer array.

For example, the 2D transducer array may include a plurality of sub-arrays, each of the plurality of sub-arrays including a plurality of transducer elements arranged in a first direction, wherein the plurality of sub-arrays are arranged in a second direction that is different from the first direction.

Furthermore, according to an embodiment, when the probe 20 is implemented as a 2D probe, the ultrasound transmitter/receiver module 110 may include at least one of an analog beamformer or a digital beamformer. Further, according to an embodiment, the 2D probe may include at least one of an analog beamformer or a digital beamformer, or a combination thereof, according to its implemented configuration.

The processor 120 may control the transmitter module 113 to form transmission signals to be respectively applied to the plurality of transducer elements based on positions and a focal point of the plurality of transducer elements.

The processor 120 may control the receiver module 117 to perform analog-to-digital conversion (ADC) on the reception signals received from the probe 20 and generate ultrasound data by summing the digital reception signals based on positions and a focal point of the plurality of transducer elements.

When the probe 20 is implemented as a 2D probe, the processor 120 may calculate a time delay value for digital beamforming with respect to each of the plurality of sub-arrays included in the 2D transducer array. Also, the processor 120 may calculate a time delay value for analog beamforming for each of the plurality of transducer elements included in any one of the plurality of sub-arrays. The processor 120 may control the analog beamformer and the digital beamformer to form a transmission signal to be applied to each of the plurality of transducer elements based on time delay values for analog beamforming and digital beamforming. The processor 120 may also control the analog beamformer to sum signals received from the plurality of transducer elements for each sub-array according to the time delay values for analog beamforming. Furthermore, the processor 120 may control the ultrasound transmitter/receiver module 110 to perform ADC on the resulting sum signal for each sub-array. In addition, the processor 120 may control the digital beamformer to generate ultrasound data by summing the digital output signals according to the time delay values for digital beamforming.

The image processor 130 generates or processes an ultrasound image by using the generated ultrasound data.

The display 140 may display the generated ultrasound image and various pieces of information processed by the ultrasound imaging apparatus 40 or the probe 20. The probe 20 or the ultrasound imaging apparatus 40 may include one or a plurality of displays 140 depending on its implemented configuration. Furthermore, the display 140 may include a touch panel or a touch screen. In addition, the display 140 may include a flexible display.

The processor 120 may control all operations of the ultrasound imaging apparatus 40 and operations of components of the ultrasound imaging apparatus 40. The processor 120 may execute programs or instructions stored in the memory 150 to perform or control various operations or functions of the ultrasound imaging apparatus 40. The processor 120 may also receive a control signal from the input interface 170 or an external device to control an operation of the ultrasound imaging apparatus 40.

The ultrasound imaging apparatus 40 includes the communication module 160 via which it may be connected to and communicate with external devices (e.g., the probe 20, servers, medical devices, and portable devices such as smartphones, tablet PCs, wearable devices, etc.).

The communication module 160 may include one or more components that enable communication with an external device. The communication module 160 may include, for example, at least one of a short-range communication module, a wired communication module, or a wireless communication module.

The communication module 160 may receive a control signal and data from an external device. The processor 120 may control an operation of the ultrasound imaging apparatus 40 in response to the control signal received via the communication module 160. Furthermore, the processor 120 may transmit a control signal to an external device via the communication module 160 to control the external device in response to the transmitted control signal. The external device may operate in response to a control signal received from the ultrasound imaging apparatus 40, or process data received from the ultrasound imaging apparatus 40.

A program or application related to the ultrasound imaging apparatus 40 may be installed on the external device. The program or application installed on the external device may control the ultrasound imaging apparatus 40, or run in response to a control signal or data received from the ultrasound imaging apparatus 40.

The external device may receive or download the program or application related to the ultrasound imaging apparatus 40 from the ultrasound imaging apparatus 40, the probe 20, or a server, and install and execute the program or application thereon. The ultrasound imaging apparatus 40, the probe 20, or the server providing a program or application may include a recording medium storing instructions, commands, installation files, executable files, or related data of the program or application. The external device may also be sold with programs or applications installed.

The memory 150 may store various types of data or programs for driving and controlling the ultrasound imaging apparatus 40, input and/or output ultrasound data, ultrasound images, etc.

The input interface 170 may receive a user input for controlling the ultrasound imaging apparatus 40. For example, the user input may include, but is not limited to, inputs for manipulating buttons, keypads, mice, trackballs, jog switches, or knops, an input for touching a touchpad or a touch screen, a voice input, a motion input, and an input of biometric information (e.g., iris recognition, fingerprint recognition, etc.).

FIG. 1B is a control block diagram of a configuration of the ultrasound imaging system 100 when the probe 20 is a wireless probe or a hybrid probe.

According to various embodiments, the ultrasound imaging apparatus 40 shown in FIG. 1B may be replaced with the ultrasound imaging apparatus 40 described with reference to FIG. 1A.

According to various embodiments, the probe shown in FIG. 1A may be replaced with the probe 20 to be described with reference to FIG. 1B.

The probe 20 may include a display 112, a transmitter module 113, a battery 114, a transducer 115, a charging module 116, a receiver module 117, an input interface 109, a processor 118, and a communication module 119. Although FIG. 1B shows that the probe 20 includes both the transmitter module 113 and the receiver module 117, according to its implemented configuration, the probe 20 may include only some of the components of the transmitter module 113 and the receiver module 117, and the ultrasound imaging apparatus 40 may also include some of the components of the transmitter module 113 and the receiver module 117. In addition, the probe 20 may further include the image processor 130.

The transducer 115 may include a plurality of transducer elements. The plurality of transducer elements are arranged in a predetermined array, forming a transducer array. The transducer array may correspond to a 1D array or a 2D array. The plurality of transducer elements may transmit ultrasound signals to an object 10 in response to transmission signals applied from the transmitter module 113. Furthermore, the plurality of transducer elements may receive ultrasound signals reflected from the object 10 to form or generate electrical reception signals.

The charging module 116 may charge the battery 114. The charging module 116 may receive power from an external source. According to an embodiment, the charging module 116 may receive power wirelessly. Furthermore, according to an embodiment, the charging module 116 may receive power by wire. The charging module 116 may transmit the received power to the battery 114.

The processor 118 may control the transmitter module 113 to generate or form transmission signals to be respectively applied to the plurality of transducer elements, based on positions and a focal point of the plurality of transducer elements.

The processor 118 may control the receiver module 117 to perform ADC on the reception signals received from the transducer 115 and generate ultrasound data by summing the digital reception signals based on positions and a focal point of the plurality of transducer elements. According to an embodiment, when the probe 20 includes the image processor 130, the image processor 130 may generate an ultrasound image based on the generated ultrasound data.

When the probe 20 is implemented as a 2D probe, the processor 118 may calculate a time delay value for digital beamforming with respect to each of the plurality of sub-arrays included in the 2D transducer array. Also, the processor 118 may calculate a time delay value for analog beamforming for each of the plurality of transducer elements included in any one of the plurality of sub-arrays. The processor 118 may control an analog beamformer and a digital beamformer to form transmission signals to be respectively applied to the plurality of transducer elements based on time delay values for analog beamforming and digital beamforming. The processor 118 may also control the analog beamformer to sum signals received from the plurality of transducer elements for each sub-array according to the time delay values for analog beamforming. Furthermore, the processor 118 may control the ultrasound transmitter/receiver module 110 to perform ADC on the resulting sum signal for each sub-array. In addition, the processor 118 may control the digital beamformer to generate ultrasound data by summing the digital output signals according to the time delay values for digital beamforming.

The processor 118 may control all operations of the probe 20 and operations of components of the probe 20. The processor 118 may execute programs or instructions stored in the memory 111 to perform or control various operations or functions of the probe 20. The processor 118 may also receive a control signal from the input interface 109 of the probe 20 or an external device (e.g., the ultrasound imaging apparatus 40) to control an operation of the probe 20. The processor 118 may also receive a control signal from the input interface 109 or an external device to control an operation of the probe 20. The input interface 109 may receive a user input for controlling the probe 20. For example, the user input may include, but is not limited to, inputs for manipulating buttons, keypads, mice, trackballs, jog switches, or knops, an input for touching a touchpad or a touch screen, a voice input, a motion input, and an input of biometric information (e.g., iris recognition, fingerprint recognition, etc.).

The display 112 may display ultrasound images generated by the probe 20, ultrasound images generated by processing ultrasound data generated by the probe 20, ultrasound images received from the ultrasound imaging apparatus 40, various pieces of information processed by the ultrasound imaging system 100, or the like. In addition, the display 112 may further display status information of the probe 20. The status information of the probe 20 may include at least one of device information of the probe 20, battery status information of the probe 20, frequency band information of the probe 20, output information of the probe 20, information about failures of the probe 20, setting information of the probe 20, or temperature information of the probe 20.

The probe 20 may include one or a plurality of displays 112 depending on its implemented configuration. Furthermore, the display 112 may include a touch panel or a touch screen. The display 112 may also include a flexible display.

The communication module 119 may wirelessly transmit the generated ultrasound data or ultrasound image to the ultrasound imaging apparatus 40 via a wireless network. The communication module 119 may also receive a control signal and data from the ultrasound imaging apparatus 40.

The ultrasound imaging apparatus 40 may receive ultrasound data or an ultrasound image from the probe 20.

In an embodiment, when the probe 20 includes the image processor 130 capable of generating an ultrasound image by using ultrasound data, the probe 20 may transmit ultrasound data or an ultrasound image generated by the image processor 130 to the ultrasound imaging apparatus 40.

In an embodiment, when the probe 20 does not include the image processor 130 capable of generating an ultrasound image by using ultrasound data, the probe 20 may transmit ultrasound data to the ultrasound imaging apparatus 40. The ultrasound data may include ultrasound raw data, and the ultrasound image may mean ultrasound image data.

The ultrasound imaging apparatus 40 may include a processor 120, an image processor 130, a display 140, a memory 150, a communication module 160, and an input interface 170.

The image processor 130 generates or processes an ultrasound image by using ultrasound data received from the probe 20.

The display 140 may display an ultrasound image received from the probe 20, an ultrasound image generated by processing ultrasound data received from the probe 20, various pieces of information processed by the ultrasound imaging system 100, or the like. The ultrasound imaging apparatus 40 may include one or a plurality of displays 140 depending on its implemented configuration. Furthermore, the display 140 may include a touch panel or a touch screen. In addition, the display 140 may include a flexible display.

The processor 120 may control all operations of the ultrasound imaging apparatus 40 and operations of components of the ultrasound imaging apparatus 40. The processor 120 may execute programs or applications stored in the memory 150 to perform or control various operations or functions of the ultrasound imaging apparatus 40. The processor 120 may also receive a control signal from the input interface 170 or an external device to control an operation of the ultrasound imaging apparatus 40.

The ultrasound imaging apparatus 40 includes the communication module 160 via which it may be connected to and communicate with external devices (e.g., the probe 20, servers, medical devices, and portable devices such as smartphones, tablet PCs, wearable devices, etc.).

The communication module 160 may include one or more components that enable communication with an external device. The communication module 160 may include, for example, at least one of a short-range communication module, a wired communication module, or a wireless communication module.

The communication module 160 of the ultrasound imaging apparatus 40 may communicate with the communication module 119 of the probe 20 by using a network or a short-range wireless communication method. For example, the communication module 160 of the ultrasound imaging apparatus 40 may communicate with the communication module 119 of the probe 20 by using any one of wireless data communication methods including a wireless local area network (WLAN), Wi-Fi, Bluetooth, ZigBee, Wi-Fi Direct (WFD), Infrared Data Association (IrDA), Bluetooth Low Energy (BLE), near field communication (NFC), wireless broadband Internet (WiBro), World Interoperability for Microwave Access (WiMAX), Shared Wireless Access Protocol (SWAP), Wireless Gigabit Alliance (WiGig), radio frequency (RF) communication, or 60 gigahertz (GHz) millimeter wave (mmWave) short-range communication.

To achieve this, the communication module 160 of the ultrasound imaging apparatus 40 and the communication module 119 of the probe 20 may each include at least one of a WLAN communication module, a Wi-Fi communication module, a Bluetooth communication module, a ZigBee communication module, a WFD communication module, an IrDA communication module, a BLE communication module, an NFC communication module, a WiBro communication module, a WiMAX communication module, a SWAP communication module, a WiGig communication module, an RF communication module, or a 60 GHz mmWave short-range communication module.

In an embodiment, the probe 20 may transmit the device information (e.g., identification (ID) information) of the probe 20 to the ultrasound imaging apparatus 40 by using a first communication method (e.g., BLE), and may be paired wirelessly with the ultrasound imaging apparatus 40. Furthermore, the probe 20 may transmit ultrasound data and/or ultrasound images to the paired ultrasound imaging apparatus 40.

The device information of the probe 20 may include various pieces of information related to a serial number, a model name, a battery status, etc. of the probe 20.

The ultrasound imaging apparatus 40 may receive, from the probe 20, the device information (e.g., ID information) of the probe 20 by using the first communication method (e.g., BLE), and may be paired wirelessly with the probe 20. Furthermore, the ultrasound imaging apparatus 40 may transmit an activation signal to the paired probe 20 and receive ultrasound data and/or ultrasound images from the probe 20. In this case, the activation signal may include a signal for controlling an operation of the probe 20.

In an embodiment, the probe 20 may transmit the device information (e.g., ID information) of the probe 20 to the ultrasound imaging apparatus 40 by using the first communication method (e.g., BLE), and may be paired wirelessly with the ultrasound imaging apparatus 40. Furthermore, by using a second communication method (e.g., 60 GHz mmWave or Wi-Fi), the probe 20 may transmit ultrasound data and/or ultrasound images to the ultrasound imaging apparatus 40 paired through the first communication method.

The ultrasound imaging apparatus 40 may receive, from the probe 20, the device information (e.g., ID information) of the probe 20 by using the first communication method (e.g., BLE), and may be paired wirelessly with the probe 20. Furthermore, the ultrasound imaging apparatus 40 may transmit an activation signal to the paired probe 20 and receive ultrasound data and/or ultrasound images from the probe 20 by using the second communication method (e.g., 60 GHz mmWave or Wi-Fi).

According to an embodiment, the first communication method used to pair the probe 20 and the ultrasound imaging apparatus 40 with each other may have a lower frequency band than the second communication method used by the probe 20 to transmit ultrasound data and/or ultrasound images to the ultrasound imaging apparatus 40.

The display 140 of the ultrasound imaging apparatus 40 may display user interfaces (UIs) indicating device information of the probe 20. For example, the display 140 may display UIs indicating ID information of the probe 20, a pairing method indicating a method of pairing the ultrasound imaging apparatus 40 with the probe 20, a status of data communication between the probe 20 and the ultrasound imaging apparatus 40, a method of performing data communication with the ultrasound imaging apparatus 40, a battery status of the probe 20, etc.

When the probe 20 includes the display 112, the display 112 of the probe 20 may display UIs indicating device information of the probe 20. For example, the display 112 may display UIs indicating ID information of the probe 20, a pairing method indicating a method of pairing the probe 20 with the ultrasound imaging apparatus 40, a status of data communication between the probe 20 and the ultrasound imaging apparatus 40, a method of performing data communication with the ultrasound imaging apparatus 40, a battery status of the probe 20, etc.

The communication module 160 may receive a control signal and data from an external device. The processor 120 may control an operation of the ultrasound imaging apparatus 40 in response to the control signal received via the communication module 160.

Furthermore, the processor 120 may transmit a control signal to an external device via the communication module 160 to control the external device in response to the transmitted control signal. The external device may operate in response to a control signal received from the ultrasound imaging apparatus 40, or process data received from the ultrasound imaging apparatus 40.

The external device may receive or download the program or application related to the ultrasound imaging apparatus 40 from the ultrasound imaging apparatus 40, the probe 20, or a server, and install and execute the program or application thereon. The ultrasound imaging apparatus 40, the probe 20, or the server providing a program or application may include a recording medium storing instructions, commands, installation files, executable files, or related data of the program or application. The external device may also be sold with programs or applications installed.

The memory 150 may store various types of data or programs for driving and controlling the ultrasound imaging apparatus 40, input and/or output ultrasound data, ultrasound images, etc.

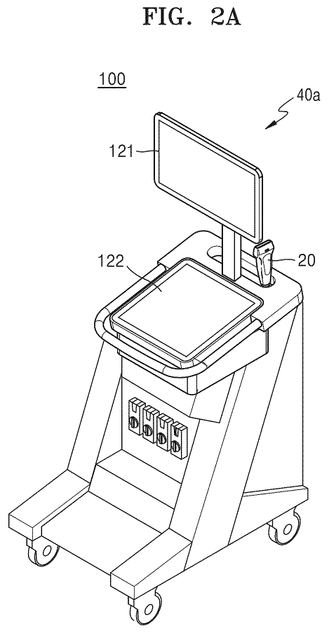

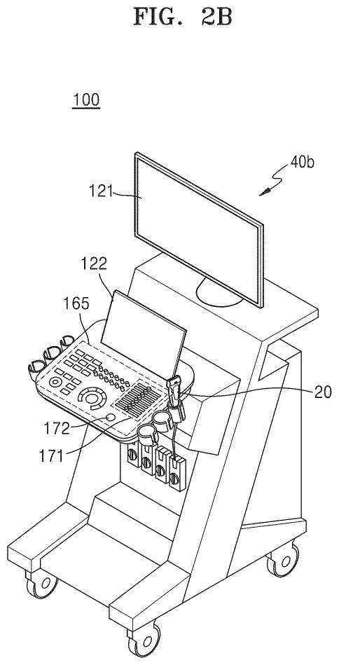

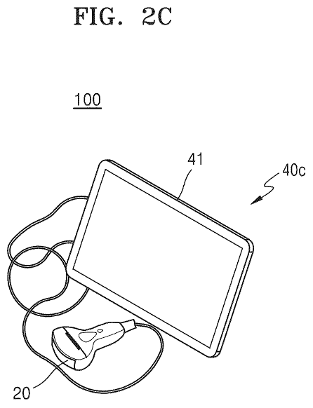

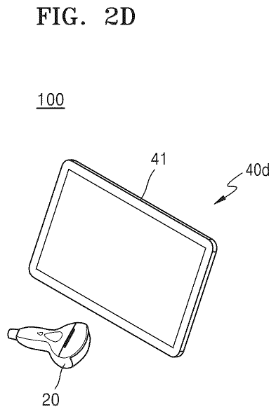

Examples of the ultrasound imaging system 100 according to an embodiment are described with reference to FIGS. 2A, 2B, 2C, and 2D.

FIGS. 2A, 2B, 2C, and 2D illustrate ultrasound imaging apparatuses according to an embodiment.

Referring to FIGS. 2A and 2B, ultrasound imaging apparatuses 40a and 40b may each include a main display 121 and a sub-display 122. The main display 121 and the sub-display 122 may correspond to the display 140 of FIGS. 1A and 1B. At least one of the main display 121 or the sub-display 122 may be implemented as a touch screen. At least one of the main display 121 or the sub-display 122 may display ultrasound images or various pieces of information processed by each of the ultrasound imaging apparatuses 40a and 40b. Furthermore, at least one of the main display 121 or the sub-display 122 may be implemented as a touch screen, and provide a graphical UI (GUI), thereby receiving, from a user, data for controlling each of the ultrasound imaging apparatuses 40a and 40b. For example, the main display 121 may display an ultrasound image, and the sub-display 122 may display a control panel for controlling display of the ultrasound image in the form of a GUI. The sub-display 122 may receive data for controlling the display of an image through the control panel displayed in the form of the GUI. For example, a time gain compensation (TGC) button, a lateral gain compensation (LGC) button, a freeze button, a trackball, a jog switch, or a knop may be provided as a GUI on the sub-display 122.

Each of the ultrasound imaging apparatuses 40a and 40b may control the display of the ultrasound image on the main display 121 by using the input control data. Furthermore, the ultrasound imaging apparatus 40a or 40b may be connected to the probe 20 by wire or wirelessly to transmit and receive ultrasound signals to and from an object.

Referring to FIG. 2B, the ultrasound imaging apparatus 40b may further include a control panel 165 in addition to the main display 121 and the sub-display 122. The control panel 165 may include buttons, trackballs, jog switches, knops, etc., and receive data for controlling the ultrasound imaging apparatus 40b from the user. For example, the control panel 165 may include a time gain compensation (TGC) button 171 and a freeze button 172. The TGC button 171 is for setting a TGC value for each depth of an ultrasound image. Also, when an input of the freeze button 172 is detected during scanning of an ultrasound image, the ultrasound imaging apparatus 40b may maintain a state in which a frame image at a corresponding time point is displayed, capture the frame image at the time point, or store the frame image at the time point.

Moreover, the buttons, trackballs, jog switches, knops, etc. included in the control panel 165 may be provided as a GUI on the main display 121 or the sub-display 122. Furthermore, the ultrasound imaging apparatus 40a or 40b may be connected to the probe 20 to transmit and receive ultrasound signals to and from the object.

Furthermore, the ultrasound imaging apparatus 40a or 40b may include various types of input/output (I/O) interfaces such as speakers, light-emitting diodes (LEDs), and vibration devices. For example, the ultrasound imaging apparatus 40a or 40b may output various pieces of information in the form of graphics, sound, or vibrations via the I/O interfaces. In addition, the ultrasound imaging apparatus 40a or 40b may output various notifications or data via the I/O interfaces.

Referring to FIGS. 2C and 2D, ultrasound imaging apparatuses 40c and 40d may also be implemented as portable ultrasound imaging apparatuses. Examples of the ultrasound imaging apparatus 40c or 40d that is portable may include, but are not limited to, a smartphone, a laptop computer, a PDA, a tablet PC, etc., each of which includes a probe and an application.

The ultrasound imaging apparatus 40c may include a main body 41. Referring to FIG. 2C, the probe 20 may be connected to one side of the main body 41 by wire. To this end, the main body 41 may include a connection terminal to or from which a cable connected to the probe 20 may be attached or detached. The probe 20 may include a cable including a connection terminal connectable to the main body 41.

Referring to FIG. 2D, the probe 20 may be wirelessly connected to the ultrasound imaging apparatus 40d. The main body 41 may include an I/O interface (e.g., a touch screen). The I/O interface may display an ultrasound image, various pieces of information processed by the ultrasound imaging apparatus 40d, a GUI, etc.

The ultrasound imaging apparatus 40d and the probe 20 may establish communication or be paired with each other by using short-range wireless communication. For example, the ultrasound imaging apparatus 40d may communicate with the probe 20 by using Bluetooth, BLE, Wi-Fi, WFD, or the like.

The ultrasound imaging apparatus 40c or 40d may execute a program or application related to the probe 20 to control the probe 20 and output information related to the probe 20. The ultrasound imaging apparatus 40c or 40d may perform operations related to the probe 20 while communicating with a certain server. The probe 20 may be registered with the ultrasound imaging apparatus 40c or 40d, or the server. The ultrasound imaging apparatus 40c or 40d may communicate with the registered probe 20 and perform operations related to the probe 20.

Furthermore, the ultrasound imaging apparatus 40c or 40d may include various types of I/O interfaces such as speakers, LEDs, and vibration devices. For example, the ultrasound imaging apparatus 40c or 40d may output various pieces of information in the form of graphics, sound, or vibrations via the I/O interfaces. In addition, the ultrasound imaging apparatus 40c or 40d may output various notifications or data via the I/O interfaces.

According to an embodiment, the ultrasound imaging apparatus 40a, 40b, 40c, or 40d may process an ultrasound image or obtain additional information from the ultrasound image by using an artificial intelligence (AI) model. According to an embodiment, by using an AI model, the ultrasound imaging apparatus 40a, 40b, 40c, or 40d may generate an ultrasound image or perform processing, such as correction, image enhancement, encoding, or decoding, on the ultrasound image. Furthermore, according to an embodiment, by using an AI model, the ultrasound imaging apparatus 40a, 40b, 40c, or 40d may perform processing, such as defining a baseline, obtaining anatomical information, obtaining lesion information, extracting surfaces, defining a boundary, measuring a length, measuring an area, measuring a volume, or generating an annotation, on an ultrasound image.

An AI model may be provided in the ultrasound imaging apparatus 40a, 40b, 40c, or 40d, or may be provided in a server.

AI models may be implemented using various artificial neural network models or deep neural network (DNN) models. Furthermore, the AI models may be trained and generated using various machine learning algorithms or deep learning algorithms. The AI models may be implemented using models such as convolutional neural networks (CNNs), recurrent neural networks (RNNs), generative adversarial networks (GANs), long short-term memory (LSTM), etc.

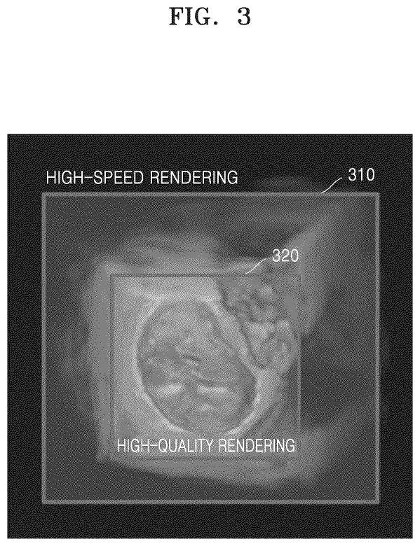

FIG. 3 illustrates a three-dimensional (3D) ultrasound image displayed by the ultrasound imaging apparatus 40, according to an embodiment.

The ultrasound imaging apparatus 40 may display a 3D ultrasound image. The ultrasound imaging apparatus 40 may obtain 3D volume data via the ultrasound transmitter/receiver module 110. The 3D volume data may be data for displaying the 3D ultrasound image. For example, the 3D volume data may be data representing a structure of the object 10, which is obtained by the ultrasound transmitter/receiver module 110.

The ultrasound imaging apparatus 40 may display a 3D ultrasound image on the display 140. The memory 150 of the ultrasound imaging apparatus 40 may store at least one instruction. The processor 120 of the ultrasound imaging apparatus 40 may be electrically connected to the ultrasound transmitter/receiver module 110, the display 140, and the memory 150.

The processor 120 may execute at least one instruction to cause the ultrasound imaging apparatus 40 to obtain 3D volume data. For example, the processor 120 may execute at least one instruction to cause the ultrasound transmitter/receiver module 110 to transmit ultrasound signals to the object 10. For example, the processor 120 may execute at least one instruction to obtain echo signals, which are ultrasound signals reflected from the object 10 and received by the ultrasound transmitter/receiver module 110. The processor 120 may obtain 3D volume data based on the echo signals.

The processor 120 may execute at least one instruction to render the 3D volume data. The processor 120 may render the 3D volume data to generate a 3D ultrasound image. The processor 120 may display the generated 3D ultrasound image on the display 140. The processor 120 may set properties for rendering the 3D volume data. For example, when rendering the 3D volume data, the processor 120 may set at least one of transparency, a color map, a gamma curve, a post-gain, or an image filter for rendering the 3D volume data.

The processor 120 may execute at least one instruction to cause the ultrasound imaging apparatus 40 to, when rendering the 3D volume data, render portions thereof for displaying different regions of the 3D ultrasound image according to different properties. For example, when rendering the 3D volume data, the processor 120 may render portions thereof for displaying different regions in the 3D ultrasound image by respectively setting at least one of different transparencies, color maps, gamma curves, post-gains, or image filters for the portions. For example, when rendering the 3D volume data, the processor 120 may render a portion thereof corresponding to a background region 310 in the 3D ultrasound image at a high speed. For example, when rendering the 3D volume data, the processor 120 may render a portion thereof corresponding to a region of interest (ROI) 320 in the 3D ultrasound image at a high quality.

The processor 120 may render the portions corresponding to the background region 310 and the ROI 320 to be distinguished from each other in real time. For example, the processor 120 may set a first density of ultrasound signals output to the background region 310 to be different from a second density of ultrasound signals output to the ROI 320. To implement this, the structure of the ultrasound transmitter/receiver module 110 may be changed. For example, at least one of a shape of a signal transmitting/receiving system of the ultrasound transmitter/receiver module 110 or a shape of an ultrasound signal output portion thereof may be changed.

The processor 120 may render the background region 310 and the ROI 320 to be distinguished from each other via post-processing. For example, the processor 120 may set a first property for post-processing the background region 310 to be different from a second property for post-processing the ROI 320. For example, the processor 120 may set at least one of a first transparency, a first color map, a first gamma curve, a first post-gain, or a first image filter for the background region 310, and may set at least one of a second transparency, a second color map, a second gamma curve, a second post-gain, or a second image filter for the ROI 320.

The ultrasound imaging apparatus 40 may render a specific region, such as the ROI 320, which needs to be displayed in more detail than the remaining regions, at a high quality. The ultrasound imaging apparatus 40 may render a region that can be relatively briefly displayed, such as the background region 310, at a high speed. The ultrasound imaging apparatus 40 may display the background region 310 and the ROI 320 in the 3D ultrasound image at different qualities by rendering the background region 310 and the ROI 320 of the 3D volume data according to different properties. The ultrasound imaging apparatus 40 may display the ROI 320 in as much detail as desired for quality while displaying the background region 310 relatively briefly, thereby improving a display speed of the 3D ultrasound image.

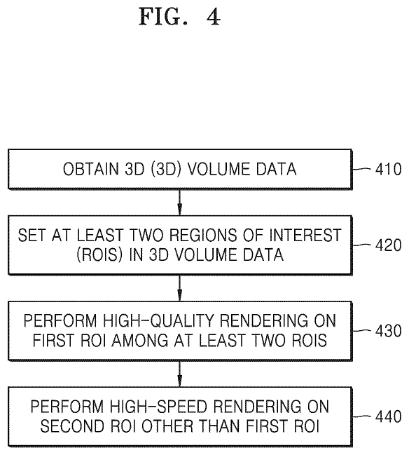

FIG. 4 is a flowchart of a method, performed by the ultrasound imaging apparatus 40, of displaying a 3D ultrasound image, according to an embodiment.

In operation 410, according to an embodiment, the ultrasound imaging apparatus 40 may obtain 3D volume data. The processor 120 of the ultrasound imaging apparatus 40 may cause the ultrasound transmitter/receiver module 110 to transmit ultrasound signals to the object 10. The processor 120 may obtain echo signals reflected from the object 10. The processor 120 may obtain 3D volume data based on the echo signals.

In operation 420, according to an embodiment, the ultrasound imaging apparatus 40 may set at least two ROIs in the 3D volume data. The processor 120 of the ultrasound imaging apparatus 40 may set an ROI in the 3D volume data to correspond to at least a region in a 3D ultrasound image. For example, the processor 120 may set an ROI in the 3D volume data to correspond to an important region in the 3D ultrasound image.

The processor 120 may set an ROI in the 3D volume data based on a preset value or a preset condition. For example, the processor 120 may set a region, which has a contrast ratio (or CR) greater than or equal to a preset value in the 3D volume data, as an ROI. For example, the processor 120 may set a region, which has a luminance greater than or equal to a preset value in the 3D volume data, as an ROI.

In operation 430, according to an embodiment, the ultrasound imaging apparatus 40 may perform high-quality rendering on a first ROI among the at least two ROIs. The processor 120 of the ultrasound imaging apparatus 40 may perform high-quality rendering on the first ROI having a greatest importance among the at least two ROIs. For example, the processor 120 may perform high-quality rendering on a region in the 3D volume data, which needs to be displayed in the most detail.

The processor 120 may perform high-quality rendering on the first ROI based on a preset value or a preset condition. For example, the processor 120 may perform high-quality rendering on the first ROI at a speed less than a preset value. For example, the processor 120 may perform high-quality rendering on the first ROI at a resolution higher than a preset value.

In operation 440, according to an embodiment, the ultrasound imaging apparatus 40 may perform high-speed rendering on a second ROI other than the first ROI. The processor 120 of the ultrasound imaging apparatus 40 may perform high-speed rendering on the second ROI having a relatively low importance compared to the first ROI. For example, the processor 120 may perform high-speed rendering on a region surrounding the region in the 3D volume data that needs to be displayed in the most detail.

The processor 120 may perform high-speed rendering on the second ROI based on a preset value or a preset condition. For example, the processor 120 may perform high-speed rendering on the second ROI at a speed greater than a preset value. For example, the processor 120 may perform high-speed rendering on the second ROI at a resolution lower than a preset value.

The processor 120 may render the first ROI and the second ROI according to different properties. The processor 120 may render the first ROI and the second ROI according to properties respectively matching the first ROI and the second ROI, thereby satisfying the image quality of a region for which a high-quality image is to be displayed. The processor 120 may render the first ROI and the second ROI according to properties respectively suitable for the first ROI and the second ROI, thereby satisfying the image display speed of a region for which a high-speed image is to be displayed.

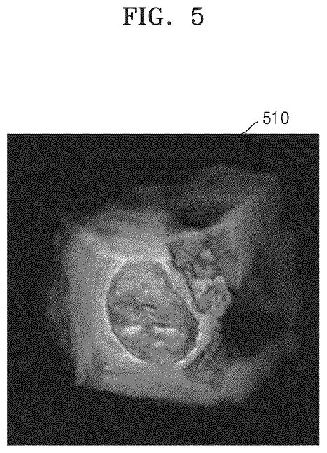

FIG. 5 is a diagram illustrating the ultrasound imaging apparatus 40 obtaining 3D volume data 510, according to an embodiment.

The processor 120 of the ultrasound imaging apparatus 40 may obtain the 3D volume data 510. The processor 120 may cause the ultrasound transmitter/receiver module 110 to transmit ultrasound signals to the object 10. The processor 120 may obtain echo signals received by the ultrasound transmitter/receiver module 110. The processor 120 may obtain the 3D volume data 510 by analyzing the echo signals. For example, when the ultrasound transmitter/receiver module 110 transmits ultrasound signals to a patient's heart, the processor 120 may analyze echo signals reflected from the patient's heart and received by the ultrasound transmitter/receiver module 110 to thereby obtain 3D volume data representing the patient's heart. For example, when the ultrasound transmitter/receiver module 110 transmits ultrasound signals to a fetus of a pregnant woman, the processor 120 may analyze echo signals reflected from the fetus of the pregnant woman and received by the ultrasound transmitter/receiver module 110 to thereby obtain 3D volume data representing the fetus.

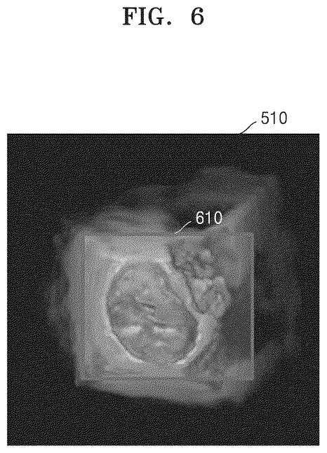

FIG. 6 is a diagram illustrating the ultrasound imaging apparatus 40 setting an ROI 610, according to an embodiment.

The processor 120 of the ultrasound imaging apparatus 40 may set at least one ROI 610 in 3D volume data 510. The processor 120 may set the ROI 610 in the 3D volume data 510 to correspond to at least a region in a 3D ultrasound image. For example, the processor 120 may set, in 3D volume data representing a patient's heart, a portion of a 3D ultrasound image showing a central portion of the patient's heart as being the ROI 610. For example, the processor 120 may set a portion of a 3D ultrasound image showing a head of a fetus as the ROI 610 in 3D volume data representing the head of the fetus.

The processor 120 may set the ROI 610 in the 3D volume data based on a preset value or a preset condition. For example, the processor 120 may set a region, which has a contrast ratio greater than or equal to a preset value in the 3D volume data, as the ROI 610. The processor 120 may determine that the region having the contrast ratio greater than or equal to the preset value indicates a region with many curves and thus contains important content. For example, the processor 120 may set a region, which has a luminance greater than or equal to a preset value in the 3D volume data, as the ROI 610. The processor 120 may determine that the region having the luminance greater than or equal to the preset value indicates a region scanned to be adjacent to a body part of the object 10 and thus contains important content.

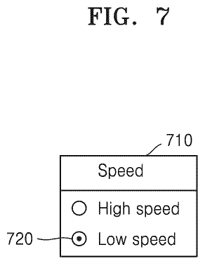

FIG. 7 is a diagram illustrating the ultrasound imaging apparatus 40 controlling a speed at which the ROI 610 is rendered, according to an embodiment.

The processor 120 of the ultrasound imaging apparatus 40 may display, on the display 140, a speed control screen 710 for controlling the speed at which the ROI 610 is rendered. The speed control screen 710 may include selection options related to the speed at which the ROI 610 is rendered. For example, the speed control screen 710 may include a first selection option for rendering the ROI 610 at a high speed and a second selection option for rendering the ROI 610 at a low speed.

The processor 120 may cause high-quality rendering to be performed on a first ROI among at least one ROI at a speed less than a preset value. The processor 120 may activate a selection menu 720 for selecting the second selection option for rendering the ROI 610 at a low speed when performing the high-quality rendering on the first ROI. The processor 120 may perform the high-quality rendering on the first ROI at a speed less than the preset value according to the activated selection menu 720.

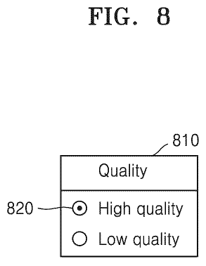

FIG. 8 is a diagram illustrating the ultrasound imaging apparatus 40 controlling a quality at which the ROI 610 is rendered, according to an embodiment.

The processor 120 of the ultrasound imaging apparatus 40 may display, on the display 140, a quality control screen 810 for controlling the quality at which the ROI 610 is rendered. The quality control screen 810 may include selection options related to the quality at which the ROI 610 is rendered. For example, the quality control screen 810 may include a first selection option for rendering the ROI 610 at a high quality and a second selection option for rendering the ROI 610 at a low quality.

The processor 120 may cause high-quality rendering to be performed on a first ROI among at least one ROI at a quality higher than a preset value. For example, the processor 120 may cause the high-quality rendering to be performed on the first ROI at a resolution higher than a preset value. The processor 120 may activate a selection menu 820 for selecting the first selection option for rendering the ROI 610 at a high quality when performing the high-quality rendering on the first ROI. The processor 120 may perform the high-quality rendering on the first ROI at a quality higher than the preset value according to the activated selection menu 820. For example, the processor 120 may perform the high-quality rendering on the first ROI at a resolution higher than the preset value.

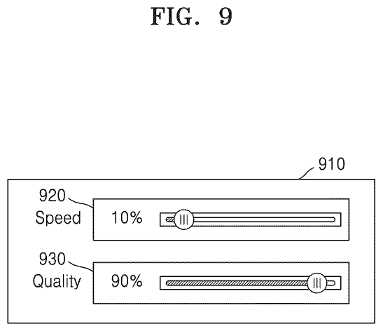

FIG. 9 is a diagram illustrating the ultrasound imaging apparatus 40 individually controlling a speed and a quality at which the ROI 610 is rendered.

The processor 120 of the ultrasound imaging apparatus 40 may display, on the display 140, an individual control screen 910 for individually controlling the speed and quality at which the ROI 610 is rendered. The individual control screen 910 may include control options related to the speed and quality at which the ROI 610 is rendered. For example, the individual control screen 910 may include a first control option 920 for controlling the speed at which the ROI 610 is rendered and a second control option 930 for controlling the quality at which the ROI 610 is rendered.

The processor 120 may individually control the speed of high-quality rendering and the resolution of the high-quality rendering for a first ROI among at least one ROI. For example, when performing the high-quality rendering on the first ROI, the processor 120 may set the first control option 920 for controlling the speed at which the ROI 610 is rendered to 10%. For example, when performing the high-quality rendering on the first ROI, the processor 120 may set the second control option 930 for controlling the quality at which the ROI 610 is rendered to 90%.

The processor 120 may perform the high-quality rendering on the first ROI at a speed set according to the first control option 920 and at a quality set according to the second control option 930. For example, the processor 120 may perform the high-quality rendering on the first ROI at a speed that is 10% of a maximum rendering speed. For example, the processor 120 may perform the high-quality rendering on the first ROI at a quality that is 90% of a maximum rendering quality.

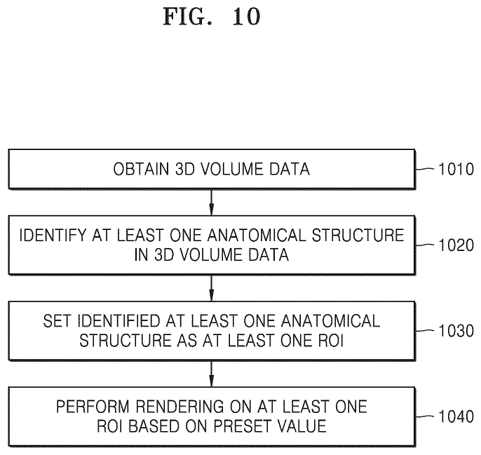

FIG. 10 is a flowchart of a method, performed by the ultrasound imaging apparatus 40, of displaying a 3D ultrasound image, according to an embodiment.

According to an embodiment, in operation 1010, the ultrasound imaging apparatus 40 may obtain 3D volume data. The processor 120 of the ultrasound imaging apparatus 40 may cause the ultrasound transmitter/receiver module 110 to transmit ultrasound signals to the object 10. The processor 120 may obtain echo signals reflected from the object 10. The processor 120 may obtain 3D volume data based on the echo signals. The 3D volume data may be data for displaying a 3D ultrasound image of the object 10. For example, the 3D volume data may be data for displaying a 3D cardiac ultrasound image of the object 10.

In operation 1020, according to an embodiment, the ultrasound imaging apparatus 40 may identify at least one anatomical structure in the 3D volume data. The anatomical structure may be a body part to be observed in the object 10. For example, the anatomical structure may be a mitral valve of the object 10. For example, the anatomical structure may be an aortic valve of the object 10. The processor 120 of the ultrasound imaging apparatus 40 may identify, in the 3D volume data, a portion corresponding to the at least one anatomical structure in a 3D ultrasound image. For example, the processor 120 may identify, in 3D volume data for displaying a 3D cardiac ultrasound image of the object 10, a portion corresponding to an aortic valve or a mitral valve in the 3D cardiac ultrasound image.

The processor 120 may identify at least one anatomical structure in the 3D cardiac ultrasound image included in the 3D volume data. For example, the processor 120 may identifya first structure including the mitral valve in the 3D cardiac ultrasound image. For example, the processor 120 may identifya second structure including the aortic valve in the 3D cardiac ultrasound image.

In operation 1030, according to an embodiment, the ultrasound imaging apparatus 40 may set the at least one identified anatomical structure as at least one ROI. The processor 120 of the ultrasound imaging apparatus 40 may set, as an ROI, the at least one anatomical structure identified in the 3D volume data. For example, the processor 120 may set, as an ROI, the first structure including the mitral valve in the 3D cardiac ultrasound image included in the 3D volume data. For example, the processor 120 may set, as an ROI, the second structure including the aortic valve in the 3D cardiac ultrasound image included in the 3D volume data.

In operation 1040, according to an embodiment, the ultrasound imaging apparatus 40 may perform rendering on the at least one ROI based on a preset value. The processor 120 of the ultrasound imaging apparatus 40 may perform rendering on the at least one ROI at a preset quality. The processor 120 may perform rendering on the at least one ROI at a preset speed. The processor 120 may perform rendering on the at least one anatomical structure set as the at least one ROI according to properties that distinguish the at least one anatomical structure from the remaining regions. For example, the processor 120 may perform high-quality rendering on an ROI corresponding to an anatomical structure that needs to be displayed in the most detail in the 3D volume data.

The processor 120 may perform rendering on each of a plurality of ROIs according to different properties. For example, the processor 120 may perform rendering on a first ROI among at least one ROI based on a first value. For example, the processor 120 may perform rendering on a second ROI, other than the first ROI, based on a second value. The processor 120 may render the first ROI and the second ROI according to properties respectively matching the first ROI and the second ROI, thereby satisfying the image quality of a region for which a high-quality image is to be displayed. The processor 120 may render the first ROI and the second ROI according to properties respectively suitable for the first ROI and the second ROI, thereby satisfying the image display speed of a region for which a high-speed image is to be displayed.

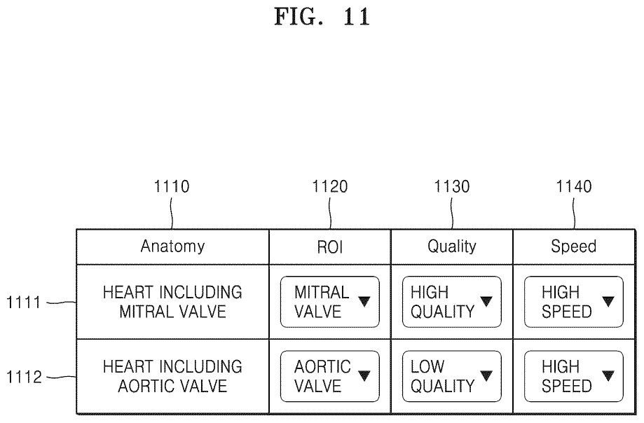

FIG. 11 is a diagram illustrating the ultrasound imaging apparatus 40 setting an anatomical structure as an ROI, according to an embodiment.

The processor 120 of the ultrasound imaging apparatus 40 may display, on the display 140, a settings screen including at least one anatomical structure (or anatomy) 1110, an ROI 1120, a quality 1130, and a speed 1140. The processor 120 may display, on the display 140, which anatomical structure the ultrasound imaging apparatus 40 has set as an ROI in a 3D ultrasound image. The processor 120 may display, on the display 140, a quality and a speed at which the ultrasound imaging apparatus 40 displays the ROI set in the 3D ultrasound image.

The processor 120 may identify the at least one anatomical structure 1110 in 3D volume data. The processor 120 of the ultrasound imaging apparatus 40 may identify, in the 3D volume data, a portion corresponding to the at least one anatomical structure in a 3D ultrasound image. For example, the processor 120 may identify a heart region including a mitral valve 1111 in 3D volume data for displaying a 3D cardiac ultrasound image of the object 10. For example, the processor 120 may identify a heart region including an aortic valve 1112 in the 3D volume data for displaying a 3D cardiac ultrasound image of the object 10.

The processor 120 may set the identified at least one anatomical structure 1110 as the at least one ROI 1120. The processor 120 may set, as the ROI 1120, the at least one anatomical structure 1110 identified in the 3D volume data. For example, the processor 120 may set, as an ROI, a first structure including the mitral valve 1111 in the 3D cardiac ultrasound image included in the 3D volume data. For example, the processor 120 may set, as an ROI, a second structure including the aortic valve 1112 in the 3D cardiac ultrasound image included in the 3D volume data.

The processor 120 may perform rendering on the at least one ROI 1120 based on a preset value. The processor 120 may perform rendering on the at least one ROI 1120 at a preset quality 1130. For example, the processor 120 may perform rendering on the first structure including the mitral valve 1111 at a high quality. For example, the processor 120 may perform rendering on the second structure including the aortic valve 1112 at a low quality. The processor 120 may perform rendering on the at least one ROI 1120 at a preset speed 1140. For example, the processor 120 may perform rendering on the first structure including the mitral valve 1111 at a high speed. For example, the processor 120 may perform rendering on the second structure including the aortic valve 1112 at a high speed.

The processor 120 may perform rendering on the at least one anatomical structure 1110 set as the at least one ROI 1120 according to properties that distinguish the at least one anatomical structure from the remaining regions. For example, the processor 120 may perform high-quality rendering on the first structure including the mitral valve 1111 that needs to be displayed in the most detail in the 3D volume data.

The processor 120 may perform rendering on each of the plurality of ROIs according to different properties. For example, the processor 120 may perform rendering on the first structure including the mitral valve 1111 from among the at least one ROI, based on a first value. For example, the processor 120 may perform rendering on the second structure including the aortic valve 1112 from among the at least one ROI, based on a second value. The processor 120 may render the first structure and the second structure according to properties respectively matching the first structure and the second structure, thereby satisfying the image quality of a region for which a high-quality image is to be displayed. The processor 120 may render the first structure and the second structure according to properties respectively suitable for the first structure and the second structure, thereby satisfying the image display speed of a region for which a high-speed image is to be displayed.

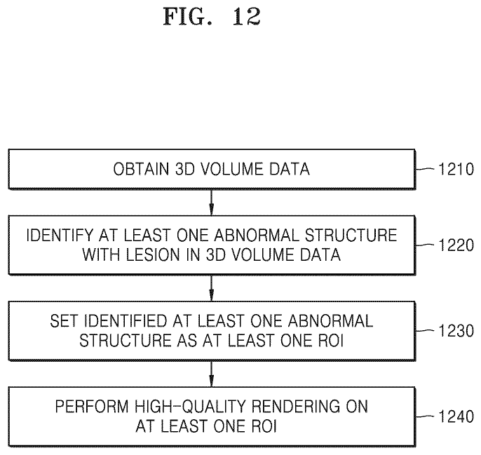

FIG. 12 is a flowchart of a method, performed by the ultrasound imaging apparatus 40, of displaying a 3D ultrasound image, according to an embodiment.

According to an embodiment, in operation 1210, the ultrasound imaging apparatus 40 may obtain 3D volume data. The processor 120 of the ultrasound imaging apparatus 40 may cause the ultrasound transmitter/receiver module 110 to transmit ultrasound signals to the object 10. The processor 120 may obtain echo signals reflected from the object 10. The processor 120 may obtain 3D volume data based on the echo signals. The 3D volume data may be data for displaying a 3D ultrasound image of the object 10. For example, the 3D volume data may be data for displaying a 3D cardiac ultrasound image of the object 10.

In operation 1220, according to an embodiment, the ultrasound imaging apparatus 40 may identify at least one abnormal structure with a lesion in the 3D volume data. The abnormal structure may be a body part including a part where the lesion occurs in the object 10. For example, the abnormal structure may be a part of the object 10 including a tumor. The processor 120 may identify, in the 3D volume data, a portion corresponding to the at least one abnormal structure in a 3D ultrasound image. For example, the processor 120 may identify, in the 3D volume data, a portion corresponding to a part including a tumor in the 3D ultrasound image.

In operation 1230, according to an embodiment, the ultrasound imaging apparatus 40 may set the identified at least one abnormal structure as at least one ROI. The processor 120 of the ultrasound imaging apparatus 40 may set, as an ROI, the at least one abnormal structure identified in the 3D volume data. For example, the processor 120 may set a portion corresponding to a part including a tumor in the 3D volume data as being an ROI.

In operation 1240, according to an embodiment, the ultrasound imaging apparatus 40 may perform high-quality rendering on the at least one ROI. The processor 120 of the ultrasound imaging apparatus 40 may perform high-quality rendering on the at least one ROI including the at least one abnormal structure. The processor 120 may perform high-quality rendering on the at least one abnormal structure that needs to be displayed in the most detail. For example, the processor 120 may perform high-quality rendering on a portion in the 3D volume data corresponding to a part including a tumor.

The processor 120 may perform rendering on an abnormal structure with a lesion and the remaining portions in the 3D volume data according to different properties. The processor 120 may render the abnormal structure and the remaining portions according to properties respectively matching the abnormal structure and the remaining portions, thereby satisfying the image quality of a portion including the abnormal structure for which a high-quality image is to be displayed. The processor 120 may render the abnormal structure and the remaining portions according to properties respectively suitable for the abnormal structure and the remaining portions, thereby satisfying the image display speed of the remaining portions for which a high-speed image is to be displayed.

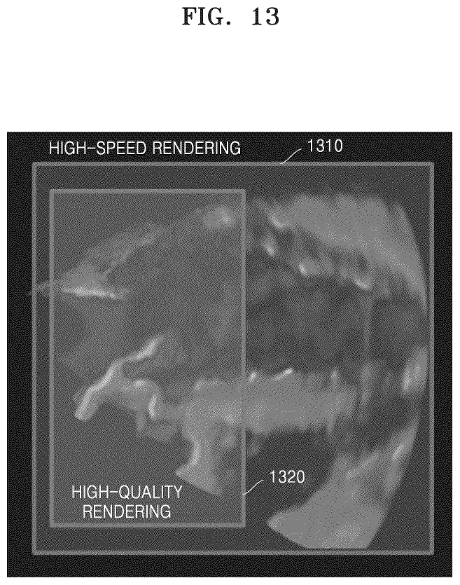

FIG. 13 is a diagram illustrating the ultrasound imaging apparatus 40 setting at least one abnormal structure 1320 as an ROI, according to an embodiment.

The processor 120 of the ultrasound imaging apparatus 40 may obtain the 3D volume data. The processor 120 may cause the ultrasound transmitter/receiver module 110 to transmit ultrasound signals to the object 10. The processor 120 may obtain echo signals reflected from the object 10. The processor 120 may obtain 3D volume data based on the echo signals. The processor 120 may display, on the display 140, a 3D ultrasound image 1310 based on the 3D volume data.

The processor 120 may identify the abnormal structure 1320 with a lesion in the 3D volume data. The abnormal structure 1320 may be a body part including a part where the lesion occurs in the object 10. For example, the abnormal structure may be a part of the object 10 including a tumor. The processor 120 may identify, in the 3D volume data, a portion corresponding to the at least one abnormal structure 1320 in a 3D ultrasound image 1310. For example, the processor 120 may identify, in the 3D volume data, a portion corresponding to a part including a tumor in the 3D ultrasound image 1310.

The processor 120 may set the identified at least one abnormal structure 1320 as at least one ROI. The processor 120 may set, as the ROI, the at least one abnormal structure 1320 identified in the 3D volume data. For example, the processor 120 may set, as the ROI, a portion in the 3D volume data corresponding to a part including a tumor.

The processor 120 may perform high-quality rendering on the at least one ROI. The processor 120 may perform high-quality rendering on the at least one ROI including the at least one abnormal structure 1320. The processor 120 may perform high-quality rendering on the at least one abnormal structure 1320 that needs to be displayed in the most detail. For example, the processor 120 may perform high-quality rendering on a portion in the 3D volume data corresponding to a part including a tumor.

The processor 120 may perform rendering on the abnormal structure 1320 with the lesion and the remaining portions in the 3D volume data according to different properties. The processor 120 may render the abnormal structure 1320 and the remaining portions according to properties respectively matching the abnormal structure and the remaining portions, thereby satisfying the image quality of a portion including the abnormal structure 1320 for which a high-quality image is to be displayed. The processor 120 may render the abnormal structure 1320 and the remaining portions according to properties respectively suitable for the abnormal structure 1320 and the remaining portions, thereby satisfying the image display speed of the remaining portions for which a high-speed image is to be displayed.

The disclosure provides an ultrasound imaging apparatus and method for displaying a 3D ultrasound image by setting properties for rendering 3D volume data and performing rendering according to the set properties in order to improve the display performance of the 3D ultrasound image.

According to an embodiment, an ultrasound imaging apparatus for displaying a 3D ultrasound image may include an ultrasound transmitter/receiver module configured to obtain 3D volume data for displaying the 3D ultrasound image, a display displaying the 3D ultrasound image, a memory storing at least one instruction, and at least one processor electrically connected to the ultrasound transmitter/receiver module, the display, and the memory, wherein the at least one processor is configured to execute the at least one instruction to cause the ultrasound imaging apparatus to obtain the 3D volume data, set at least two ROIs in the 3D volume data, perform high-quality rendering on a first ROI among the at least two ROIs, and perform high-speed rendering on a second ROI other than the first ROI.

According to an embodiment, the at least one processor may execute the at least one instruction to cause the ultrasound imaging apparatus to set a region, which has a contrast ratio greater than or equal to a preset value in the 3D volume data, as one of the at least two ROIs.

According to an embodiment, the at least one processor may execute the at least one instruction to cause the ultrasound imaging apparatus to set a region, which has a luminance greater than a preset value in the 3D volume data, as one of the at least two ROIs.

According to an embodiment, the at least one processor may execute the at least one instruction to cause the ultrasound imaging apparatus to perform the high-quality rendering on the first ROI at a speed less than a preset value.

According to an embodiment, the at least one processor may execute the at least one instruction to cause the ultrasound imaging apparatus to perform the high-quality rendering on the first ROI at a resolution higher than a preset value.

According to an embodiment, the at least one processor may execute the at least one instruction to cause the ultrasound imaging apparatus to perform the high-speed rendering on the second ROI at a higher speed than used for the high-quality rendering.

According to an embodiment, the at least one processor may execute the at least one instruction to cause the ultrasound imaging apparatus to individually control a speed of the high-quality rendering and a resolution of the high-quality rendering.

According to an embodiment, a method, performed by an ultrasound imaging apparatus, of displaying a 3D ultrasound image may include obtaining 3D volume data, setting at least two ROIs in the 3D volume data, performing high-quality rendering on a first ROI among the at least two ROIs, and performing high-speed rendering on a second ROI other than the first ROI.

According to an embodiment, the setting of the at least two ROIs may include setting a region, which has a contrast ratio greater than or equal to a preset value in the 3D volume data, as one of the at least two ROIs.

According to an embodiment, the setting of the at least two ROIs may include setting a region, which has a luminance greater than a preset value in the 3D volume data, as one of the at least two ROIs.

According to an embodiment, the performing of the high-quality rendering may include performing the high-quality rendering on the first ROI at a speed less than a preset value.

According to an embodiment, the performing of the high-quality rendering may include performing the high-quality rendering on the first ROI at a resolution higher than a preset value.

According to an embodiment, the performing of the high-speed rendering may include performing the high-speed rendering on the second ROI at a higher speed than used for the high-quality rendering.

According to an embodiment, the performing of the high-quality rendering may include individually controlling a speed of the high-quality rendering and a resolution of the high-quality rendering.

According to an embodiment, a method, performed by an ultrasound imaging apparatus, of displaying a 3D ultrasound image may include obtaining 3D volume data, identifying at least one anatomical structure in the 3D volume data, setting the identified at least one anatomical structure as at least one ROI, and performing rendering on the at least one ROI based on a preset value.

According to an embodiment, the 3D volume data may include a 3D cardiac ultrasound image, and the identifying of the at least one anatomical structure may include identifying a first structure including a mitral valve and a second structure including an aortic valve in the 3D cardiac ultrasound image.

According to an embodiment, the performing of the rendering may include performing rendering on a first ROI among the at least one ROI based on a first value, and performing rendering on a second ROI, other than the first ROI, based on a second value.

According to an embodiment, the identifying of the at least one anatomical structure may include identifying at least one abnormal structure having a lesion in the 3D volume data.

According to an embodiment, the setting as the at least one ROI may include setting the identified at least one abnormal structure as the at least one ROI.

According to an embodiment, the performing of the rendering may include performing high-quality rendering on the at least one ROI.

According to the disclosure, the performance of displaying a 3D ultrasound image may be improved by focusing on a quality at which some regions within a single 3D ultrasound image are rendered and focusing on a speed of rendering for the other regions.

According to the disclosure, by improving the rendering quality with respect to an ROI, a part that needs to be represented in detail, such as a lesion, may be displayed clearly.

According to the disclosure, by increasing the speed at which the remaining regions are rendered, a 3D ultrasound image may be efficiently utilized for a part where rapid movement needs to be observed, such as the heart of an object.

An apparatus, method, or computer program according to an embodiment performs operations related to AI. Operations related to AI are performed via a processor and a memory. The processor may use one or a plurality of processors to perform operations related to AI. In this case, the one or plurality of processors may be a general-purpose processor such as a central processing unit (CPU), an application processor (AP), a digital signal processor (DSP), etc., a dedicated graphics processor such as a graphics processing unit (GPU), a vision processing unit (VPU), etc., or a dedicated AI processor such as a neural processing unit (NPU). The one or the plurality of processors process input data according to programs, instructions, or AI models stored in the memory.

AI-related programs, instructions, or AI models may be created via machine learning. In this case, the creation via the machine learning means that the programs, instructions, or AI models designed to perform desired characteristics (or purposes) are created by training base AI models based on a large number of training data via learning algorithms. The machine learning may be performed by an apparatus itself in which AI is performed or via a separate server and/or system. Examples of a learning algorithm may include, but are not limited to, supervised learning, unsupervised learning, semi-supervised learning, and reinforcement learning.

An AI model may consist of a plurality of neural network layers. Each of the plurality of neural network layers has a plurality of weight values and performs neural network computations via calculations between a result of computations in a previous layer and the plurality of weight values. The plurality of weight values assigned to each of the plurality of neural network layers may be optimized by a result of training the AI model. An artificial neural network may include a deep neural network (DNN), and may be, for example, but is not limited to, a CNN, a DNN, an RNN, a restricted Boltzmann machine (RBM), a deep belief network (DBN), a bidirectional recurrent DNN (BRDNN), or deep Q-networks (DQNs).

A machine-readable storage medium may be provided in the form of a non-transitory storage medium. In this regard, the term ‘non-transitory’ only means that the storage medium does not include a signal (e.g., an electromagnetic wave) and is a tangible device, and the term does not differentiate between where data is semi-permanently stored in the storage medium and where the data is temporarily stored in the storage medium. For example, the ‘non-transitory storage medium’ may include a buffer in which data is temporarily stored.

According to an embodiment, methods according to embodiments may be included in a computer program product when provided. The computer program product may be traded, as a product, between a seller and a buyer. The computer program product may be distributed in the form of a machine-readable storage medium (e.g., compact disc read only memory (CD-ROM)) or distributed (e.g., downloaded or uploaded) on-line via an application store or directly between two user devices (e.g., smartphones). For online distribution, at least a part of the computer program product (e.g., a downloadable app) may be at least transiently stored or temporally generated in a machine-readable storage medium such as a memory of a server of a manufacturer, a server of an application store, or a relay server.

What is claimed is:

1. An ultrasound imaging apparatus for displaying a three-dimensional (3D) ultrasound image, the ultrasound imaging apparatus comprising:
an ultrasound transmitter/receiver module configured to obtain 3D volume data for displaying the 3D ultrasound image;
a display displaying the 3D ultrasound image;
a memory storing at least one instruction; and
at least one processor electrically connected to the ultrasound transmitter/receiver module, the display, and the memory, wherein
the at least one processor is configured to execute the at least one instruction to cause the ultrasound imaging apparatus to
obtain the 3D volume data,
set at least two regions of interest (ROIs) in the 3D volume data,
perform high-quality rendering on a first ROI among the at least two ROIs, and
perform high-speed rendering on a second ROI other than the first ROI.

2. The ultrasound imaging apparatus of claim 1, wherein the at least one processor is further configured to execute the at least one instruction to cause the ultrasound imaging apparatus to set a region, which has a contrast ratio greater than or equal to a preset value in the 3D volume data, as one of the at least two ROIs.

3. The ultrasound imaging apparatus of claim 1, wherein the at least one processor is further configured to execute the at least one instruction to cause the ultrasound imaging apparatus to set a region, which has a luminance greater than or equal to a preset value in the 3D volume data, as one of the at least two ROIs.

4. The ultrasound imaging apparatus of claim 1, wherein the at least one processor is further configured to execute the at least one instruction to cause the ultrasound imaging apparatus to perform the high-quality rendering on the first ROI at a speed less than a preset value.

5. The ultrasound imaging apparatus of claim 1, wherein the at least one processor is further configured to execute the at least one instruction to cause the ultrasound imaging apparatus to perform the high-quality rendering on the first ROI at a resolution higher than a preset value.

6. The ultrasound imaging apparatus of claim 1, wherein the at least one processor is further configured to execute the at least one instruction to cause the ultrasound imaging apparatus to perform the high-speed rendering on the second ROI at a higher speed than used for the high-quality rendering.

7. The ultrasound imaging apparatus of claim 1, wherein the at least one processor is further configured to execute the at least one instruction to cause the ultrasound imaging apparatus to individually control a speed of the high-quality rendering and a resolution of the high-quality rendering.

8. A method, performed by an ultrasound imaging apparatus, of displaying a three-dimensional (3D) ultrasound image, the method comprising:
obtaining 3D volume data;
setting at least two regions of interest (ROIs) in the 3D volume data;
performing high-quality rendering on a first ROI among the at least two ROIs; and
performing high-speed rendering on a second ROI other than the first ROI.

9. The method of claim 8, wherein the setting of the at least two ROIs comprises setting a region, which has a contrast ratio greater than or equal to a preset value in the 3D volume data, as one of the at least two ROIs.

10. The method of claim 8, wherein the setting of the at least two ROIs comprises setting a region, which has a luminance greater than or equal to a preset value in the 3D volume data, as one of the at least two ROIs.

11. The method of claim 8, wherein the performing of the high-quality rendering comprises performing the high-quality rendering on the first ROI at a speed less than a preset value.

12. The method of claim 8, wherein the performing of the high-quality rendering comprises performing the high-quality rendering on the first ROI at a resolution higher than a preset value.

13. The method of claim 8, wherein the performing of the high-speed rendering comprises performing the high-speed rendering on the second ROI at a higher speed than used for the high-quality rendering.

14. The method of claim 8, wherein the performing of the high-quality rendering comprises individually controlling a speed of the high-quality rendering and a resolution of the high-quality rendering.

15. A method, performed by an ultrasound imaging apparatus, of displaying a three-dimensional (3D) ultrasound image, the method comprising:
obtaining 3D volume data;
identifying at least one anatomical structure in the 3D volume data;
setting the identified at least one anatomical structure as at least one region of interest (ROI); and
performing rendering on the at least one ROI based on a preset value.

16. The method of claim 15, wherein
the 3D volume data includes a 3D cardiac ultrasound image, and
the identifying of the at least one anatomical structure comprises
identifying a first structure including a mitral valve and a second structure including an aortic valve in the 3D cardiac ultrasound image.

17. The method of claim 15, wherein
the performing of the rendering comprises:
performing rendering on a first ROI among the at least one ROI based on a first value; and
performing rendering on a second ROI, other than the first ROI, based on a second value.

18. The method of claim 15, wherein the identifying of the at least one anatomical structure comprises identifying at least one abnormal structure having a lesion in the 3D volume data.

19. The method of claim 18, wherein the setting as the at least one ROI comprises setting the identified at least one abnormal structure as the at least one ROI.

20. The method of claim 19, wherein the performing of the rendering comprises performing high-quality rendering on the at least one ROI.
